(19) United States
(12) Patent Application Publication
(10) Pub. No.: US 2026/0016492 A1
(43) Pub. Date: Jan. 15, 2026

(54) POINT-OF-CARE BLOOD ANALYSIS SYSTEM

(71) Applicant: Hylid Diagnostics Inc., Ottawa (CA)

(72) Inventors: Nigel John FAIRBANK, Ottawa (CA); Sape DE VRIES, Ottawa (CA); John Berryhill WALKER, Ottawa (CA)

(21) Appl. No.: 18/995,961

(22) PCT Filed: Jul. 18, 2023
(86) PCT No.: PCT/CA2023/050967

§ 371 Date: Jan. 17, 2025

Related U.S. Application Data
(60) Provisional application No. 63/390,376, filed on Jul. 19, 2022.

(51) Int. Cl. G01N 33/72; G01N 21/03; G01N 21/64; G01N 33/50; G01N 33/68

(52) U.S. Cl. CPC G01N 33/721; G01N 21/03; G01N 21/645; G01N 33/5002; G01N 33/68; G01N 2021/0325

(57) ABSTRACT

The present disclosure relates to a point-of-care blood analysis system. specifically to the disposable. single-use test cartridges used in such a system. The test cartridge is inexpensive and simple to use because it contains neither a calibration fluid pack nor a separate sensor substrate. The test cartridge accepts a whole blood sample and then separates plasma from a portion of that whole blood sample. The test cartridge has broad application because it allows analytes in both the whole blood and the separated plasma to be measured using both fluorescent sensors and cuvettes.

### CROSS REFERENCE TO RELATED APPLICATIONS

The application claims priority and the benefit of U.S. Provisional Patent Application Ser. No. 63/390,376, entitled “POINT-OF-CARE BLOOD ANALYSIS SYSTEM” filed on Jul. 19, 2022, the disclosure of which is incorporated herein by reference in its entirety. The application is also a national phase entry of International Patent Application No. PCT/CA2023/050967, entitled “POINT-OF-CARE BLOOD ANALYSIS SYSTEM” filed on Jul. 18, 2023, the disclosures of which is incorporated herein by reference in its entirety.

### BACKGROUND

The embodiments described herein pertain to the field of fluid analysis systems, in particular point-of-care blood analysis systems.

### Point-of-Care Blood Analysis

Point-of-care (POC) blood analysis systems are medical devices that perform in vitro blood analysis in close proximity to the patient (in locations such as a physician's office, the patient's home, or a remote ward of a hospital) rather than in a large, dedicated blood-testing laboratory relatively far from the patient. POC blood testing shortens the time to results primarily by eliminating the transportation of the patient and/or the blood sample to the lab. This allows a more rapid clinical response to test results, which is particularly valuable in acute care and major surgery. It also allows a uniformity of care that is independent of the location of blood-testing laboratories. Moreover, a number of analytes can actually change in concentration with time and transportation, resulting in measurements less reflective of the original sample.

POC blood analysis systems typically comprise a portable instrument and single-use, disposable test cartridges containing one or more sensors. Each sensor typically measures one analyte of interest in a discrete blood sample applied to the cartridge. The volume of the blood sample is typically 100 microliters or less. POC blood analysis is primarily done using electrochemical sensors and occasionally done using fluorescent sensors.

Some applications—such as the monitoring of chronic kidney disease (CKD)—require measuring multiple analytes with relatively small allowable errors. In practice, these allowable errors are generally set by the Clinical Laboratory Improvement Amendments of 1988 (CLIA) regulations, which include federal standards applicable to all U.S. facilities or sites that test human specimens for health assessment or to diagnose, prevent, or treat disease. To date, this achieving acceptable levels of error has necessitated a level of system cost and complexity that has made multi-analyte measuring systems—such as the Abbott i-STAT and, Siemens epoc, Siemens RAPIDPoint 500, and Radiometer ABL90—effectively inaccessible outside clinical settings. There is a need for a simpler, less expensive alternative that can perform lab-accurate measurements outside clinical settings, and ideally when operated by patients themselves.

### Whole Blood Versus Plasma

Whole blood—i.e., blood in vivo or an unaltered blood sample in vitro—consists primarily of four components: plasma, red blood cells, white blood cells and platelets. Plasma is the liquid component and normally has a pale yellow colour.

Sensors, whether electrochemical or optical, typically react only to fluids diffusing into the sensor. Sensors therefore typically only respond to the plasma component of whole blood.

Plasma may be obtained by removing the red blood cells, white blood cells and platelets from whole blood. For our purposes, we will use the term plasma to indicate whole blood from which at least the red blood cells have been removed or extracted.

Hemolysis and its Effect on Analytes such as Potassium

Hemolysis is the rupture of red blood cells. Hemolysis can be caused by the act of drawing a blood sample, whether a venous draw using a needle or a fingerstick draw using a lancet. With a fingerstick draw, hemolysis can be exacerbated by “milking” or squeezing the finger in order to elicit blood flow.

Hemolysis can change the concentration of analytes in plasma. One analyte that is particularly affected by hemolysis is potassium (K). The concentration of K inside red blood cells is approximately 20X-30X higher than the concentration of K in plasma. Hemolysed red blood cells release this high concentration of K into the plasma which raises the concentration of K in the plasma. Since plasma is the component that is measured by sensors, this can lead to falsely elevated K measurements. Even a small percentage of hemolysed red blood cells in a blood sample can lead to spuriously high K measurements. This effect is common enough that it has a name, pseudo-hyperkalemia, meaning false high potassium.

Pseudo-hyperkalemia can be detected by measuring the degree of hemolysis in the blood sample. Since hemoglobin (Hb) is normally found only inside red blood cells, the presence of free Hb in plasma (i.e., plasma free hemoglobin, pfHb) indicates hemolysis. Plasma can be separated from the whole blood, then the plasma pfHb concentration measured via characteristic absorption by hemoglobin of light incident on the plasma. The pfHb alert levels set by typical central lab analyzers is in the range of 0.25-1 mg/dl as discussed in Lippi G, Salvagno GL, Blanckaert N, et al. Multicenter evaluation of the hemolysis index in automated clinical chemistry systems. Clin Chem Lab Med. 2009: 47 (8): 934-939. Excessive pfHb levels require that the test be re-run on a new blood sample.

Accordingly, there is a need in the art for POC test cartridges capable of accepting a whole blood sample, measuring analytes (such as K) in that whole blood sample, then separating plasma from that same whole blood sample, and measuring pfHb in the separated plasma. It is critical that plasma derives from exactly the same volume of blood that was used to measure the K, so that any indication of hemolysis pertains directly to the K measurement. Such a test cartridge would enable certain analyte measurements (such as K) to move out of the central laboratory without sacrificing the pfHb measurement that is crucial to clinical trustworthiness and decision-making.

### Plasma Separation

Some analytes are optimally measured in whole blood and some analytes are optimally measured in plasma.

Diagnostic scenarios exist that require the measurement of one or more analytes in whole blood and also one or more analytes in plasma. Obtaining plasma from a whole blood sample is difficult in the POC setting where the user lacks both the equipment (such as a centrifuge) and the skill required to separate plasma from whole blood. Furthermore, performing plasma separation incorrectly can cause hemolysis (e.g., centrifuging for too long).

Accordingly, there is a need in the art for POC test cartridges capable of accepting a whole blood sample, measuring analytes (such as K) in that whole blood sample, then separating plasma from that same whole blood sample, and then measuring analytes (such as pfHb) in the separated plasma. Performing all these steps in the test cartridge relieves the user from having to prepare both a whole blood sample and a plasma sample. The simplicity that results from having all steps performed inside a single test cartridge is a key factor to enabling such measurements in the home or outside of a clinical environment.

### Optical Measurement: Fluorescent Sensors

Fluorescent sensors contain one or more fluorescent probes chosen so that the excitation spectrum and/or the emission spectrum varies with the concentration of the analyte. The analyte concentration can thus be determined by collecting the appropriate spectra and analyzing them relative to spectra from reference samples.

### Optical Measurement: Cuvettes

Cuvettes are chambers filled with a sample fluid used to measure the transmission loss of light passing through the sample fluid. The cuvettes themselves, and the materials from which they're made, generally have low transmission losses relative to the sample fluid. Typical cuvette materials include clear, transparent glass and clear, transparent polymers such as PETG, PMMA and COC.

Transmission losses due to the sample fluid are effectively due to one of two factors: light is absorbed by materials in the sample fluid (either directly by the analyte itself or by reagents added to the sample fluid) or light is scattered by materials in the sample fluid (typically particles such as cells). The change in transmission, referenced to transmission prior to sample introduction, correlates with the concentration of the analyte.

### Calibration

Sensors are typically calibrated at the time of sensor manufacturing. Such factory calibration converts the measured sensor output (for example, intensity of the emission spectrum) to units needed by the user (for example, analyte concentration in mg/dL).

In order to achieve clinically-acceptable (or ‘lab-quality’) accuracy and precision for given analytes—in practice, to conform with the total allowable error requirements as set by CLIA and the Clinical & Laboratory Standards Institute (CLSI)—today's POC blood analysis systems (whether using electrochemical sensors or fluorescent sensors) typically require another sensor calibration immediately prior to use. Such time-of-use calibration corrects for variations in sensor analyte response since the factory calibration.

Time-of-use calibration is typically performed using a calibration fluid incorporating analytes to be measured at known concentrations. The calibration fluid is housed in a sealed calibration fluid pack, often on the test cartridge itself. The calibration fluid and sample must be separately measured, close enough in time for the sample measurement to be sufficiently accurate. In practice, the calibration fluid is often measured just prior to the sample, after controlled release from the calibration fluid pack and onto the sensors. Then the sample must be measured within a few minutes.

Sensors that do not need time-of-use calibration would result in a simpler and cheaper test cartridge (not requiring the calibration fluid pack components), a simpler and cheaper instrument (not requiring a mechanism to release the calibration fluid), and a simpler workflow for the user (neither having to wait for the time-of-use calibration process to be completed nor having a window of time after which that time-of-use calibration is invalidated).

### Separate Sensor Substrate

Electrochemical sensors generate electrical signals which must be conducted to the instrument for measurement. Signal conduction is typically achieved by fabricating the electrochemical sensors onto a separate substrate incorporating conducting elements and then adding this separate substrate to the test cartridge. Diagnostic test cartridges incorporating separate electrochemical sensor substrates require numerous parts and assembly steps during test cartridge manufacturing.

Fluorescent sensors may also be fabricated on a separate substrate. For fluorescent sensors, a separate substrate consisting chiefly of a porous matrix (such as cellulose) can facilitate optimal printing, bonding and drying of the sensors. Diagnostic test cartridges incorporating separate fluorescent sensor substrates require numerous parts and assembly steps during test cartridge manufacturing.

There is a desire to fabricate sensors directly onto the test cartridge without the use of a separate sensor substrate which would result in a simple, inexpensive test cartridge.

### SUMMARY

An object of the present disclosure is to provide a test cartridge for a POC blood analysis system. The test cartridge is capable of accepting a whole blood sample, separating plasma from that whole blood sample, and measuring analytes in both the whole blood and in the separated plasma. The test cartridge allows analytes in both the whole blood and in the separated plasma to be measured via fluorescent sensors and via transmission losses in cuvettes.

The test cartridge is capable of measuring analytes (such as K) in the whole blood sample, then moving that same whole blood sample to a plasma separator, then separating plasma from that same whole blood sample, and then measuring analytes (such as pfHb) in the separated plasma. Using the same blood sample for all measurements minimizes the volume of blood required to be provided by the user. Measuring all analytes on the same blood sample ensures that, for example, the hemolysis measurement correlates directly with the potassium measurement.

The fluorescent sensors do not require time-of-use calibration, which eliminates the need for a calibration fluid pack. Elimination of the calibration fluid pack reduces the cost and complexity of the test cartridge.

The fluorescent sensors are printed directly onto the body of the test cartridge, which eliminates the need for a separate sensor substrate. Elimination of the separate sensor substrate reduces the cost and complexity of the test cartridge.

There is also provided an instrument for determining the concentration of analytes in a sample fluid. The instrument comprises a source of excitation light for the fluorescent sensors and a means to measure the intensity of the excitation light and/or the emitted fluorescent light across multiple wavelengths. The instrument also comprises a source of light for transmission through the cuvettes, and a means to measure the intensity of the transmitted light across multiple wavelengths after it has passed through the cuvette. The instrument includes a computer processing means to convert the measured light intensities to analyte concentrations using proprietary mathematical models.

There is also provided software on a mobile device that will securely accept information (measurement results) transmissions from the instrument. Furthermore, the software on the mobile device will facilitate the secure transmission of information to the patient's electronic medical record (EMR) so that test records may be shared with the patient's physician.

### BRIEF DESCRIPTION OF THE DRAWINGS

These and other features of the disclosure will become more apparent in the following detailed description in which reference is made to the appended drawings.

FIG. 1A is a diagram illustrating an exemplary test cartridge.

FIG. 1B is a diagram illustrating the exemplary test cartridge with the top and bottom labels removed.

FIG. 1C is a diagram illustrating an exploded view of the exemplary test cartridge.

FIG. 1D is a diagram illustrating a cross-sectional view of the plasma separation region of the exemplary test cartridge.

FIG. 2A is a diagram illustrating a further exemplary test cartridge.

FIG. 2B is a diagram illustrating the further exemplary test cartridge with the top and bottom labels removed.

FIG. 2C is a diagram illustrating an exploded view of the further exemplary test cartridge.

FIG. 2D is a diagram illustrating an exploded view of the plasma separation module of the further exemplary test cartridge.

FIG. 2E is a diagram illustrating a cross-sectional view of the plasma separation region of the further exemplary test cartridge.

FIG. 3A is a diagram illustrating an exemplary test cartridge that measures only K and pfHb.

FIG. 3B is a diagram illustrating the K and pfHb test cartridge with the top and bottom labels removed.

FIG. 3C is a diagram illustrating an exploded view of the exemplary K and pfHb test cartridge.

FIG. 3D is a diagram illustrating an exploded view of the plasma separation module of the exemplary K and pfHb test cartridge.

FIG. 3E is a diagram illustrating a cross-sectional view of the plasma separation region of the K and pfHb test cartridge.

FIG. 4A is a diagram illustrating measurement data from the fluorescent K sensor.

FIG. 4B is a diagram illustrating a successful plasma separation performed in-house using the perimeter blood-loading technique.

FIG. 4C is a diagram illustrating measurement data for pfHb concentration vs absorbance from a cuvette filled with plasma.

FIG. 4D is a diagram illustrating measurement data from the fluorescent pH sensor lab blood analyzer in the same whole blood sample

FIG. 4E is a diagram illustrating measurement data for tHb concentration versus absorbance in whole blood samples.

### DETAILED DESCRIPTION

The present disclosure relates to a POC blood analysis system, specifically to the disposable, single-use test cartridges used in such a system. The test cartridge is inexpensive and simple to use because it contains neither a calibration fluid pack nor a separate sensor substrate. The test cartridge accepts a whole blood sample and separates plasma from that whole blood sample. The test cartridge has broad application because it allows analytes in both the whole blood and the separated plasma to be measured via fluorescent sensors and via transmission losses in cuvettes.

### Fluorescent Sensors

In accordance with an aspect of the present disclosure, there is provided a fluorescent sensor, containing one or more fluorescent probes chosen so that the excitation spectrum and/or the emission spectrum varies with the concentration of the analyte of interest.

The excitation and/or fluorescent emission spectra are measured with sufficient precision across a sufficiently broad wavelength range so that a mathematical model can be constructed that converts the measured data into analyte concentrations meeting the required accuracy and precision.

To help meet the required accuracy and precision, factory test data could be used in the mathematical model. The factory test data could be stored on either the test cartridge itself or on its packaging. Data storage could be done by means known in the art such as 1D barcodes, 2D barcodes, RFID tags and so forth.

The measurement of the spectra across a sufficiently broad wavelength range, the sophistication of the mathematical model, the use of factory test data in that mathematical model, and the relative stability of fluorescent sensors all help contribute to sensors that do not need time-of-use calibration to deliver the required accuracy and precision. Eliminating the calibration fluid pack reduces the complexity and cost of the test cartridge.

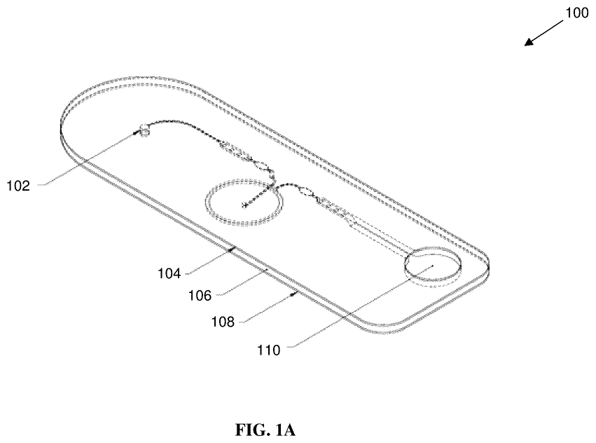

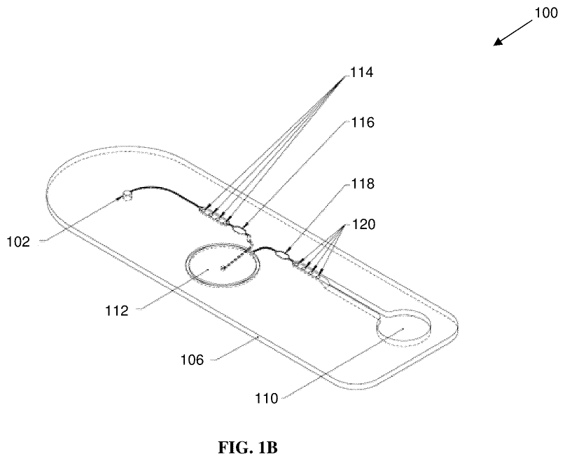

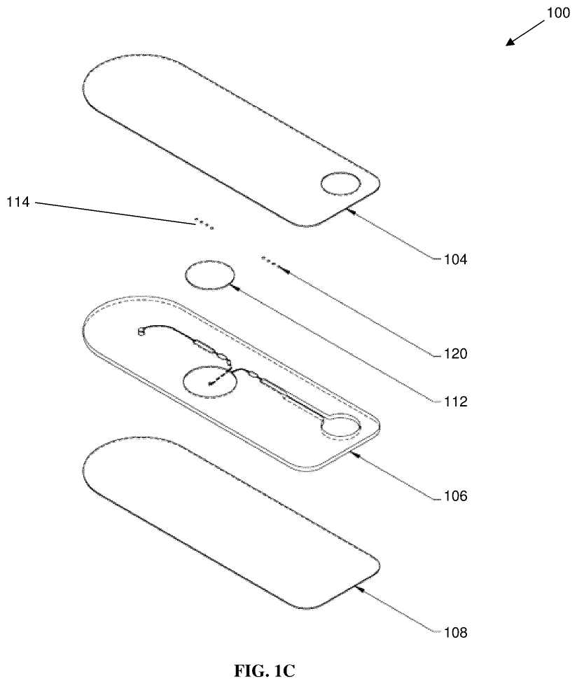

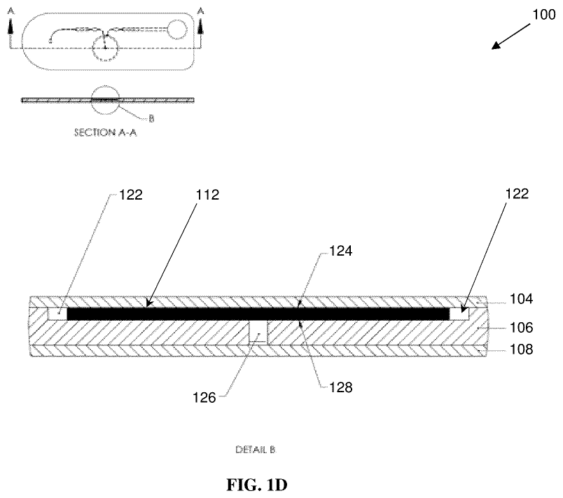

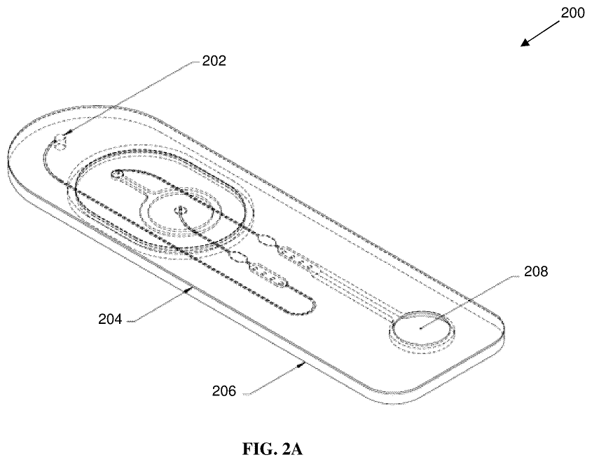

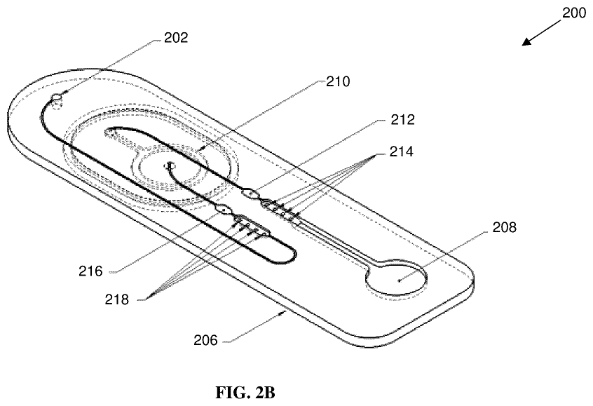

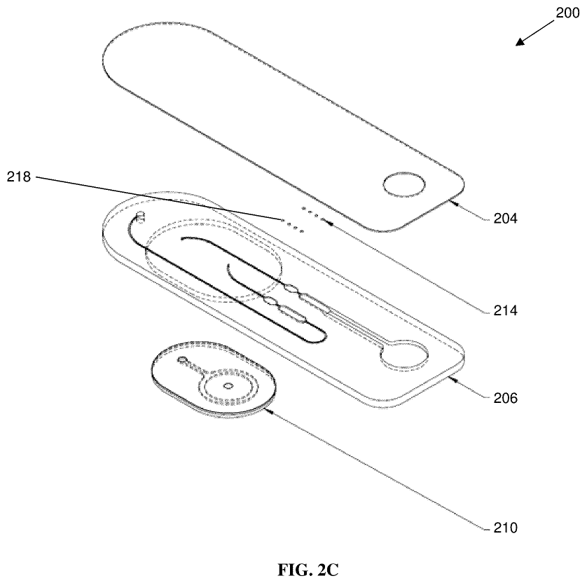

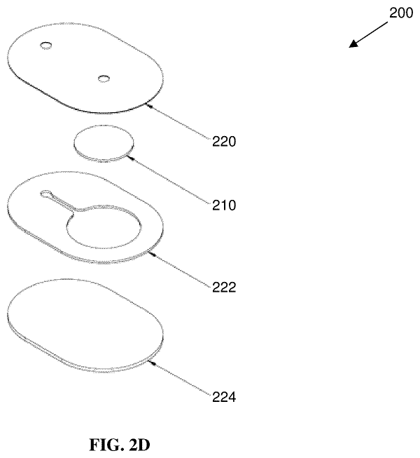

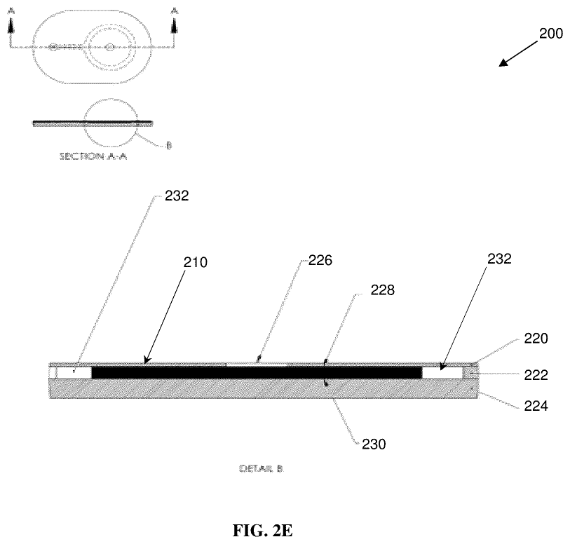

There is also provided a method of fluorescent sensor manufacturing. The fluorescent sensor is first created as a suspension and then a small volume of this suspension is deposited or ‘printed’ directly onto the body of the test cartridge. FIGS. 1B and 2B illustrate possible print locations in channels on the body of the test cartridge. The printed material is then dried to a certain extent; the drying process may be entirely passive or may be accelerated with heat and/or a reduction of the ambient relative humidity or pressure. The sensor may be constructed of one or more separate layers, with the layers printed on top of one another, each not necessarily covering the layer below it completely.

The manufacturing technique of printing the fluorescent sensors directly onto the body of the test cartridge avoids the need to first print the fluorescent sensors onto a separate sensor substrate and then cutting, aligning and bonding the separate sensor substrate to the test cartridge. This reduces the complexity and cost of the test cartridge.

### Test Cartridge

In accordance with another aspect of the disclosure, there is provided a single-use test cartridge, or ‘consumable’, for optical measurement of analytes in a sample fluid.

FIGS. 1A-1D illustrate the test cartridge design when plasma separation is done using a plasma separation membrane and this membrane is directly bonded to the body of the test cartridge (bonding can be done by any means known in the art, including ultra-sonic welding and pressure-sensitive adhesives).

FIG. 1A is a diagram illustrating an exemplary test cartridge. According to FIG. 1A, test cartridge 100 comprises air vent 102, top label 104, body 106, bottom label 108 and sample port 110. Air vent 102 provides a connection to atmosphere or to a pump in the instrument.

FIG. 1B is a diagram illustrating the exemplary test cartridge with the top and bottom labels removed. According to FIG. 1B, test cartridge 100 is shown with air vent 102, body 106 and sample port 110. Furthermore, test cartridge 100 also contains plasma separation membrane 112, a plurality of printed fluorescence sensors 114 housed in a plasma channel, cuvette 116 in the plasma channel, cuvette 118 in a whole blood channel and printed fluorescent sensors 120 in the whole blood channel.

FIG. 1C is a diagram illustrating an exploded view of the exemplary test cartridge. According to FIG. 1C, test cartridge 100 comprises top label 104, body 106 and bottom label 108. Sandwiched between top label 104 and body 106 is plasma separation membrane 112 and fluorescence sensors 114 and 120.

FIG. 1D is a diagram illustrating a cross-sectional view of the plasma separation region of the exemplary test cartridge. According to FIG. 1D, test cartridge 100 has a top label 104, body 106, bottom label 108 and plasma exit 126.

According to FIG. 1D, whole blood first fills the circular channel around the perimeter of the plasma separation membrane at 122 and then moves between the plasma separation membrane 112 and the top label 104. Furthermore, the blood side of the plasma separation membrane 124 is not bonded to top label 104. According to FIG. 1D, the plasma side of plasma separation membrane 128 is bonded to the body 106 of the test cartridge 100. This bonding can be achieved using ultrasonic welding and/or other mechanical bonding techniques.

FIGS. 2A-2E illustrate the test cartridge design when plasma separation is done using a plasma separation membrane and this membrane is contained in a plasma separation module. This module is bonded to the body of the test cartridge (bonding can be done by any means known in the art, including pressure-sensitive adhesives).

FIG. 2A is a diagram illustrating a further exemplary test cartridge. According to FIG. 2A, further exemplary test cartridge 200 comprises air vent 202, label 204 (or top label), body 206 and sample port 208. Air vent 202 provides a connection to atmosphere or to a pump in the instrument.

FIG. 2B is a diagram illustrating the further exemplary test cartridge with the top and bottom labels removed. According to FIG. 2B, test cartridge 200 comprises of air vent 202, body 206, sample port 208 and plasma separation module 210. Further to FIG. 2B, test cartridge 200 further comprises cuvette 212 in whole blood channel, printed fluorescent sensors 214 in whole blood channel, cuvette 216 in plasma channel and printed fluorescent sensors 218 in plasma channel.

FIG. 2C is a diagram illustrating an exploded view of the further exemplary test cartridge. According to FIG. 2C, test cartridge 200 comprises top label 204, body 206 and plasma separation module 210. Plasma separation module 210 is placed beneath (or under) body 206. Sandwiched between top label 204 and body 206 are fluorescence sensors 214 and 218.

FIG. 2D is a diagram illustrating an exploded view of the plasma separation module of the further exemplary test cartridge. According to FIG. 2D, test cartridge 200 is shown in exploded view having plasma via layer 220, membrane space layer 222 and closing layer 224. Plasma separation membrane 210 is found sandwiched between plasma via layer 220 and closing layer 224.

According to FIG. 2D, plasma via layer 220 further comprises a polymer film, a PSA on top face to bond to the cartridge body, a PSA on the bottom face to bond to the plasma separation membrane 210 and to the membrane spacer layer 222.

According to FIG. 2D, plasma separation membrane 210 further comprises a top face bonded to the PSA on the bottom face of the plasma via layer 220 and a bottom face touching, but not bonded to, the closing layer 224.

According to FIG. 2D, membrane spacer layer 222 further comprises a polymer film, no PSA on the top face but bonded to the PSA on the bottom face of the plasma via layer 220 and a PSA on the bottom face to bond to the closing layer 224. Closing layer 224 further comprises a polymer film with no PSA at all so that it does not bond to the plasma separation membrane 210, but bonded to the PSA on the bottom face of the membrane spacer layer 222.

FIG. 2E is a diagram illustrating a cross-sectional view of the plasma separation region of the further exemplary test cartridge. According to FIG. 2E, test cartridge 200 has a plasma via layer 220, membrane spacer layer 222, closing layer 224, plasma separation membrane 210 and plasma via 226. The plasma via 226 is where plasma exits.

According to FIG. 2E, whole blood first fills the circular channel around the perimeter of the plasma separation membrane at 232 and then moves between the plasma separation membrane 210 and closing layer 224 which is not bonded to the plasma separation membrane 210.

According to FIG. 2E, the plasma side of plasma separation membrane 228 is bonded to the plasma via layer 220 and the blood side of the plasma separation membrane 230 is not bonded to the closing layer 224.

FIGS. 1A and 2A illustrate two possible designs of the test cartridge. The test cartridge comprises a body made using techniques known in the art, such as injection molding using any of a number of polymers includes PETG, PMMA and COC. The test cartridge contains a sample port where the sample fluid is applied. FIGS. 1A, 1B, 2A and 2B illustrate possible locations of the sample port. The volume of whole blood required is approximately 25 μl (though other volumes may be used).

In some embodiments, the test cartridge contains a hydrophilic element so that sample fluid applied to the sample port is drawn into the test cartridge through capillary force, to an extent greater than it would be without the hydrophilic element. The test cartridge contains one or more fluidic channels to convey the sample fluid from the sample port to the various elements within the test cartridge.

In some embodiments, the test cartridge contains fluorescent sensors. FIGS. 1B and 2B illustrate possible locations of fluorescent sensors. Examples of analytes measured via these fluorescent sensors could include potassium (K), pH, and calcium (Ca).

In some embodiments, the test cartridge contains cuvettes. FIGS. 1B and 2B illustrate possible locations of cuvettes. Examples of analytes measured via light transmission loss in cuvettes could include plasma free hemoglobin (pfHb), total hemoglobin (tHb), bilirubin (Bili), creatinine (Crea) and hematocrit (Hct). In some embodiments, the cartridge is designed so that the light source is located on one side of the cartridge and the means to measure the intensity of the transmitted light is located on the other side of the cartridge, so that the light passes through the cuvette once. In this embodiment, we measure transmitted light. In some further embodiments, the cartridge is designed so that the light source is located on the same side of the cartridge as the means to measure the intensity of the transmitted light. In this embodiment, the light passes through the cuvette a first time, reverses direction by reflecting off a surface such as a reflective label, and then passes through the cuvette a second time. In this embodiment, we measure reflected light.

In some embodiments, the test cartridge contains a plasma separation element to remove blood cells from whole blood. When the sample fluid is whole blood, the fluid upstream of the plasma separation element is whole blood and the fluid downstream of the plasma separation element is plasma.

In some embodiments, the test cartridge contains fluorescent sensors and cuvettes upstream of the plasma separation element and also contains fluorescent sensors and cuvettes downstream of the plasma separation element. FIGS. 1B and 2B illustrate possible locations of fluorescent sensors and cuvettes both upstream and downstream of the plasma separation element. In this manner, the test cartridge allows the measurement of analytes in whole blood via fluorescent sensors and via transmission losses in cuvettes, and also the measurement of analytes in plasma via fluorescent sensors and via transmission losses in cuvettes.

In some embodiments, the test cartridge contains fluorescent sensors only upstream or only downstream of the plasma separation element. In some embodiments, the test cartridge contains cuvettes only upstream or only downstream of the plasma separation element.

In some embodiments, the plasma separation element is a plasma separation membrane. FIGS. 1B and 2D illustrate possible locations of the plasma separation membrane. The plasma separation membrane acts as a filter that traps blood cells while allowing plasma to flow through the filter. The plasma separation membrane may be constructed from materials that include glass fibers, cellulose acetate and polymers such as polysulfone. The plasma separation membrane is typically porous with hole sizes that are smaller than the size of blood cells; in this manner, the membrane may be used to filter out blood cells. Whole blood enters the membrane on one side, referred to as the blood-side surface, and plasma exits the membrane from the other side, referred to as the plasma-side surface.

In some embodiments, the plasma separation membrane may be loaded with reagents that enable certain measurements in the separated plasma. In some embodiments, the membrane pore sizes may be larger than the size of blood cells and the membrane may also be loaded with reagents; in such an embodiment, the membrane no longer separates plasma but serves only to add reagents to the whole blood sample and thereby enable certain measurements in the whole blood. One example of such a reagent is a hemolyzing agent that could hemolyze the whole blood as it flows through the membrane; one could then measure total hemoglobin (tHb) by way of a transmission measurement in a cuvette. In some embodiments, reagents could be added to the membrane to bind specific blood components to the membrane itself for measurement of those components. One example of such a reagent is tagged antibodies that change their optical properties when they bind to certain proteins. In further embodiments, the membrane in combination with reagents can be expanded to other uses above and beyond plasma separation.

The two cartridge designs illustrated in FIGS. 1A-1D, and the cartridges illustrated in FIGS. 2A-2E accomplish plasma separation in similar ways, differing only in the method of manufacture. The cartridge design illustrated in FIGS. 1A-1D adds the plasma separation membrane directly to the body of the cartridge; in this design, the cartridge manufacturing process includes a step where the plasma separation membrane is bonded to the body of the cartridge by any means known in the art, including ultrasonic welding and pressure sensitive adhesive. The cartridge design illustrated in FIGS. 2A-2E uses a pre-manufactured module that contains the plasma separation membrane and adds this module to the body of the cartridge. The module contains the plasma separation membrane and 3 polymer layers. These elements may be bonded to one another by any means known in the art, including sensitive adhesives. The module may be pre-manufactured by a supplier and delivered in a format conducive to automated manufacturing, such as a reel. Reeled modules closely resemble the reeled labels that are well known in the art and may permit the use of off-the-shelf label placing equipment to peel the modules from the reel and to place the modules into the body of the cartridge. As with labels, the face of the module that mates to the body of the cartridge may be covered in a pressure-sensitive adhesive.

In some embodiments, the plasma separation region is designed to allow the movement of the whole blood onto substantially the entire blood-side surface of the plasma separation membrane before plasma separation substantially begins. This design intent is the same in the cartridge illustrated in FIGS. 1A-1D as it is in the cartridge illustrated in FIGS. 2A-2E. If the whole blood is moved onto just a small portion of the blood-side surface of the plasma separation membrane, then that small portion may quickly clog with red blood cells, stopping the plasma separation process prematurely. Furthermore, any portion of the plasma separation membrane not filled with whole blood remains empty and tends to soak up separated plasma, preventing that separated plasma from leaving the plasma separation membrane and being used elsewhere in the cartridge. Therefore, movement of the whole blood onto substantially the entire blood-side surface of the plasma separation membrane before separation begins is required for successful plasma separation.

In some embodiments, movement of the whole blood onto substantially the entire blood-side surface of the plasma separation membrane is enabled by a circular channel surrounding the perimeter of the plasma separation membrane, as well as a cover layer that touches, but is not bonded to, the blood-side surface of the plasma separation membrane. This geometry is illustrated in FIGS. 1D and 2E. The circular channel has a large cross-sectional area and allows fast flow of the whole blood, while the negligible gap between the blood-side surface of the plasma separation membrane and the cover layer has a negligibly small cross-sectional area and does not allow fast flow of the whole blood. In this manner, whole blood entering the plasma separation region first substantially fills the entire circular channel before flowing between the blood-side surface of the plasma separation membrane and the cover layer. The whole blood first substantially surrounding the entire perimeter of the plasma separation membrane ensures that the whole blood wets the blood-side surface of the plasma separation membrane from all directions and results in full wetting of substantially the entire blood-side surface of the plasma separation membrane.

This design confers a number of benefits. The first benefit is that very little, if any, of the plasma separation membrane remains unfilled with whole blood. This means that separated plasma is not wasted by soaking into any unfilled portions of the plasma separation membrane, which minimises the volume of blood required to successfully run the test cartridge. Minimising the volume of the blood sample that needs to be collected from the user is important as this increases the ease of use of the product. A second benefit of filling the plasma separation membrane using a circular channel around the perimeter of the plasma separation membrane, rather than over the blood-side surface of the plasma separation membrane, is that this design adds zero thickness to the cartridge, resulting in a thin cartridge that minimises the complexity of the design. FIG. 2D illustrates the simplicity of using just 3 layers to create the entire plasma separation module: a spacer layer in the middle (the same thickness as the plasma separation membrane) that creates the circular channel around the perimeter of the plasma separation membrane and then a layer on each side. The simplicity of the design minimises the number of cartridge components and minimises manufacturing complexity, which reduces the cartridge cost.

In some embodiments, the movement of the sample fluid through the test cartridge is accomplished without applying any external pressure, i.e., passively, through capillary and gravitational forces alone, for example. Capillary force may be enhanced with elements such as hydrophilic films or hydrophilic membranes, which could form part or all of the walls of a fluidic channel. Capillary force could also be enhanced by altering the surface chemistry of the test cartridge body, for example through plasma treatment, to render it more hydrophilic. These methods of capillary force enhancement can be done individually or in combination.

The test cartridge contains one or more air vents in fluidic connection with the sample fluid channel to vent the air displaced by the sample fluid. FIGS. 1A and 2A illustrate possible locations of the air vent. The test cartridge may contain a vent element, such as a hydrophobic porous plug, that allows air to pass but contains fluid inside the test cartridge.

In some embodiments, the flow speed of the whole blood into the circular channel may need to be kept above a certain minimum flow speed. Plasma separation membranes can exhibit significant capillary draw. A strong capillary draw may cause the whole blood to begin to flow between the blood-side surface of the plasma separation membrane and its cover layer before the whole blood has substantially surrounded the entire perimeter of the plasma separation membrane. In such cases, speeding up the flow of whole blood in the circular channel ensures that the whole blood substantially surrounds the entire perimeter of the plasma separation membrane before the whole blood flows significantly between the blood-side surface of the plasma separation membrane and the cover layer. Speeding up the flow rate of the whole blood in the circular channel may be accomplished by, for example, the use of negative pressure applied downstream of the plasma separation module. To effect this, the instrument may be provided with a pumping system that connects to the test cartridge upon insertion of the test cartridge into the instrument.

In some embodiments, the movement of the sample fluid through the test cartridge is aided by application of external pressure, such as with a pumping system in the instrument. The test cartridge contains one or more fluidic channels that connect to the pumping system in the instrument. In some embodiments, the air vent is the element that connects to the pumping system in the instrument. The pumping system may apply negative pressure, or vacuum, to pull the whole blood into the test cartridge, and/or move the whole blood over the whole blood fluorescent sensors and into the whole blood cuvette, and/or move the whole blood into the plasma separation element, and/or help draw the separated plasma away from the plasma separation element, and/or move the separated plasma into the plasma cuvette and over the plasma fluorescent sensors. Alternately, the pumping system may apply positive pressure to push the sample fluid, resulting in the same outcome. The test cartridge may contain a vent element, such as a hydrophobic porous plug, that allows air to pass but contains fluid inside the test cartridge.

In some embodiments, the test cartridge, or its packaging, contains a means to store information. The information stored may include regulatory information such as test cartridge serial number, lot number, expiry date and unique device identifier (UDI) code. The information stored may also include factory test data such as measured calibration curves for the fluorescent sensors and measured optical path lengths for the cuvettes. The means of information storage are known in the art and include printed 1D barcodes, printed 2D barcodes, RFID tags and so forth.

In some embodiments, the test cartridge contains a label on one or both faces. FIGS. 1A and 2A illustrate possible locations of labels. The labels may be used to close certain fluidic channels and may also contain printed regulatory information such as test cartridge serial number, lot number, expiry date and UDI code.

### Instrument

In accordance with another aspect of the disclosure, there is provided an instrument for determining the concentration of analytes in a sample fluid.

In some embodiments, the device is configured to accept the test cartridges of the disclosure.

In some embodiments, the instrument comprises a source of excitation light for the fluorescent sensors, a method to collect the emitted fluorescent light and a means to measure the intensity of the emitted fluorescent light across a certain wavelength range, providing either a single intensity measurement, or multiple measurements by wavelength, the latter resulting in an intensity spectrum and being accomplished by a device such as a spectrometer. The instrument also comprises a source of light for transmission through the cuvettes, a method to collect the transmitted or reflected light and a means to measure the intensity of the transmitted or reflected light across a certain wavelength range.

In some embodiments the instrument comprises a means to read information stored on the test cartridge. The means of information reading are known in the art and include an optical barcode reader and a RFID tag reader.

In some embodiments the instrument comprises a pumping system, including an air pump, optical sensors and pneumatic valves. The instrument comprises a means to connect the pumping system to the test cartridge. The pumping system may apply negative pressure to pull the whole blood into the test cartridge, and/or to move the whole blood into the plasma separation element, and/or to help draw the separated plasma away from the plasma separation element. The optical sensors are used to detect the arrival of whole blood and plasma at key locations within the test cartridge; this information is used to trigger certain actions of the pumping operation. The pneumatic valves are used to switch the actions of the pumping operation from one mode to another; the modes may include venting the test cartridge to atmosphere, connecting the test cartridge to the air pump, and isolating the test cartridge from the air pump.

In some embodiments the instrument comprises a computer processing means to convert the measured light intensities to analyte concentrations using proprietary mathematical models. These proprietary mathematical models may make use of any factory test data that may be stored on the test cartridge such as measured factory calibration curves for the fluorescent sensors and measured optical path lengths for the cuvettes.

In some embodiments the instrument comprises a screen to display information to the user; examples of the information that may be displayed include instrument status, instructions for use, measured analyte concentrations from the current test, and summaries of measured analyte concentrations from past tests. In some embodiments the screen may be a touch screen allowing the user to enter information via an on-screen keyboard; examples of the information that may be entered include patient details and physician details.

In some embodiments the instrument comprises a means to transmit test records to software on a mobile device such as a mobile phone or a tablet computer. The means for transmission from the instrument to the device include methods known in the art such as Bluetooth and wi-fi. The screen of the mobile device may be used instead of or in addition to, the instrument screen to display and enter information.

### Software

In some embodiments the software on the mobile device will securely accept test record transmissions from the instrument. Furthermore, the software on the mobile device will facilitate the secure transmission of those test records to the patient's electronic medical record (EMR) so that test records may be shared with the patient's physician.

In some embodiments the software on the mobile device will include an interface allowing the physician to contact and communicate with the patient; such communication may be accomplished with a number of means known in the art, including texting, voice calling and video calling.

To gain a better understanding of the disclosure described herein, the following examples are set forth. It will be understood that these examples are intended to describe illustrative embodiments of the disclosure and are not intended to limit the scope of the disclosure in any way.

Test Cartridge with Potassium Sensor and Hemolysis Detection Example

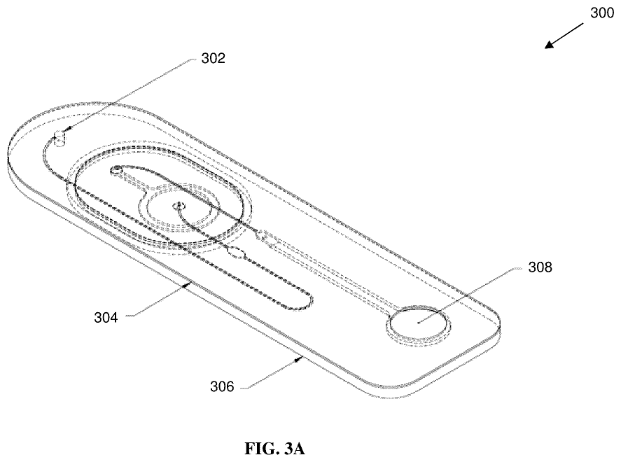

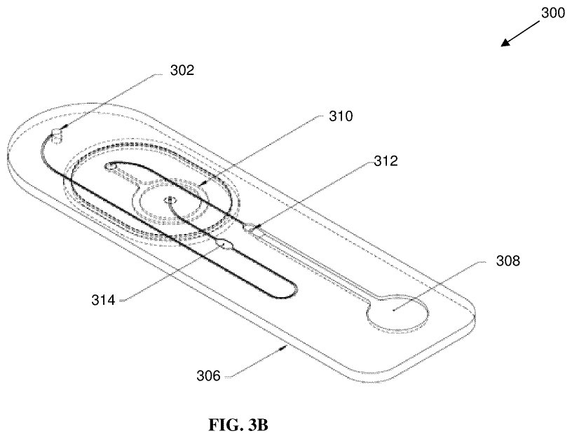

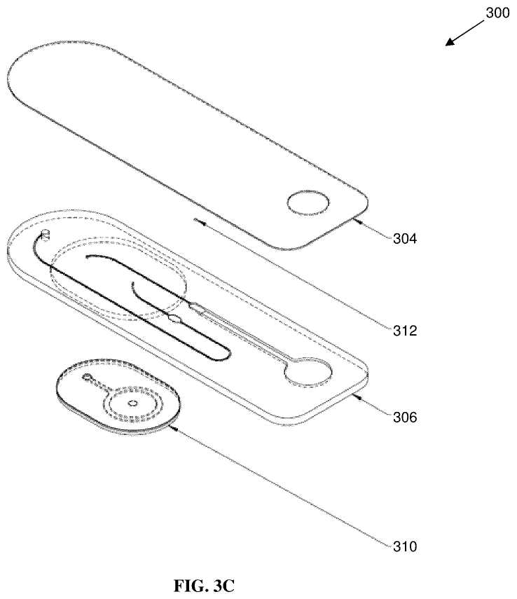

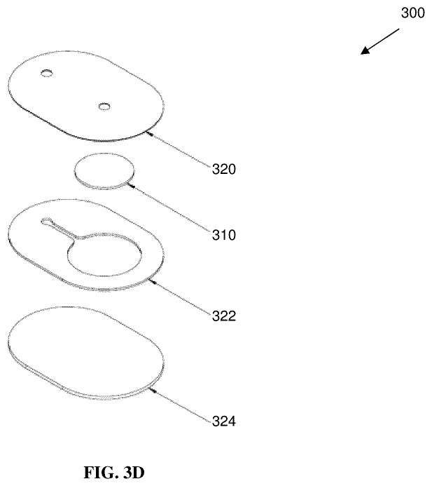

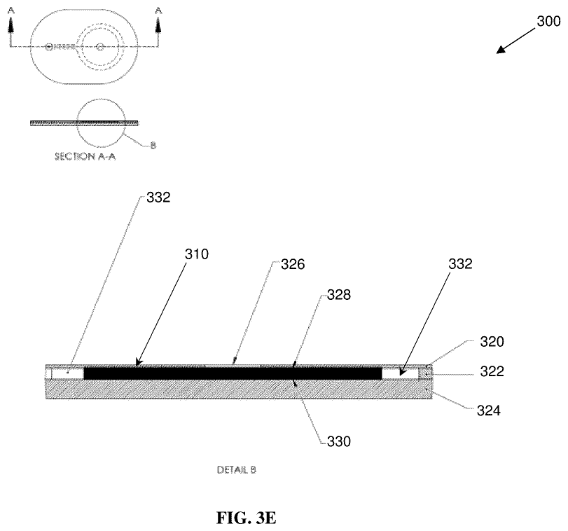

In accordance with an aspect of the present disclosure, there is provided a test cartridge for the optical measurement of K in a whole blood sample and the optical measurement of pfHb in plasma separated from that same whole blood sample. FIGS. 3A-3E illustrate such a test cartridge.

FIGS. 3A-3E illustrate a test cartridge design for the example of a test cartridge that measures only K and pfHb. In this example, there is only one fluorescent sensor in whole blood, for the K measurement, and there is no need for a cuvette in whole blood. In this example, there is a cuvette in plasma, for the pfHb measurement, and there is no need for any fluorescent sensors in plasma. Therefore, this example represents a simplified version of the test cartridge illustrated in FIGS. 2A-2E. In a similar manner, one could simplify the test cartridge illustrated in FIGS. 1A-1D for the same example of a test cartridge that measures only K and pfHb.

FIG. 3A is a diagram illustrating an exemplary test cartridge that measures only K and pfHb. According to FIG. 3A, a further exemplary test cartridge 300 comprises air vent 302, label 304 (or top label), body 306 and sample port 308. Air vent 302 provides a connection to the pumping system in the instrument.

FIG. 3B is a diagram illustrating the K and pfHb test cartridge with the top and bottom labels removed. According to FIG. 3B, test cartridge 300 comprises an air vent 302, body 306, sample port 308 and plasma separation module 310. Further in FIG. 3B, test cartridge 300 further comprises printed fluorescent K sensor 312 in whole blood channel and cuvette for pfHb measurement 314 in plasma channel.

FIG. 3C is a diagram illustrating an exploded view of the exemplary K and pfHb test cartridge. According to FIG. 2C, test cartridge 300 comprises top label 304, body 306 and plasma separation module 310. Plasma separation module 310 is placed beneath (or under) body 306. Sandwiched between top label 304 and body 306 is fluorescence K sensor 312.

FIG. 3D is a diagram illustrating an exploded view of the plasma separation module of the exemplary K and pfHb test cartridge. According to FIG. 3D, test cartridge 300 is shown in exploded view having plasma via layer 320, membrane space layer 322 and closing layer 324. Plasma separation membrane 310 is found sandwiched between plasma via layer 320 and closing layer 324.

According to FIG. 3D, plasma via layer 320 further comprises a polymer film, a PSA on top face to bond to the cartridge body, a PSA on the bottom face to bond to the plasma separation membrane 310 and to the membrane spacer layer 322.

According to FIG. 3D, plasma separation membrane 310 further comprises a top face bonded to the PSA on the bottom face of the plasma via layer 320 and a bottom face touching, but not bonded to, the closing layer 324.

According to FIG. 2D, membrane spacer layer 322 further comprises a polymer film, no PSA on the top face but bonded to the PSA on the bottom face of the plasma via layer 320 and a PSA on the bottom face to bond to the closing layer 324. Furthermore, closing layer 324 further comprises a polymer film with no PSA at all so that it does not bond to the plasma separation membrane 310, but bonded to the PSA on the bottom face of the membrane spacer layer 322.

FIG. 3E is a diagram illustrating a cross-sectional view of the plasma separation region of the K and pfHb test cartridge. According to FIG. 3E, test cartridge 300 has a plasma via layer 320, membrane spacer layer 322, closing layer 324, plasma separation membrane 310 and plasma via 326. Plasma via 326 is where the plasma exits.

According to FIG. 3E, whole blood first fills the circular channel around the perimeter of the plasma separation membrane at 332 and then moves between the plasma separation membrane 310 and closing layer 324 which is not bonded to the plasma separation membrane 310.

According to FIG. 2E, the plasma side of plasma separation membrane 328 is bonded to the plasma via layer 320 and the blood side of the plasma separation membrane 330 is not bonded to the closing layer 324.

The test cartridge comprises a body made using techniques known in the art, such as injection molding using any of a number of polymers includes PETG, PMMA and COC. The test cartridge contains a sample port where the sample fluid is applied. FIGS. 3A and 3B illustrate possible locations of the sample port. The volume of whole blood required is approximately 25 μl (though other volumes may be used).

The test cartridge contains a single fluidic channel to convey the sample fluid from the sample port to the various elements within the test cartridge. The test cartridge may contain a hydrophilic element so that sample fluid applied to the sample port is drawn into the test cartridge through capillary force, to an extent greater than it would be without the hydrophilic element. The hydrophilic element could be a label made with a hydrophilic material or coated with a hydrophilic pressure-sensitive adhesive.

The test cartridge contains an air vent at the end of the single fluidic channel that may be used to vent the air displaced by the whole blood sample when it enters the test cartridge. FIGS. 3A and 3B illustrate the possible location of the air vent. The test cartridge may contain a vent element, such as a hydrophobic porous plug, that allows air to pass but contains fluid inside the test cartridge.

The test cartridge contains a fluorescent K sensor in the whole blood channel, as illustrated in FIG. 3B. The fluorescent sensor contains one or more fluorescent probes chosen so that the excitation spectrum and/or the emission spectrum varies with the concentration of the K in the plasma component of the whole blood sample. The excitation and/or fluorescent emission spectra are measured with sufficient precision across a sufficiently broad wavelength range so that a mathematical model converts the measured spectral data into analyte concentrations meeting the required accuracy and precision.

The spectral data used to determine analyte concentration could either be a single spectrum at a given point in time after application of the sample, or multiple spectra at different points in time. For example, sensor emission spectra taken 2 minutes after sample application—and averaged over a period of 1 second to reduce noise—could be divided by similarly time—averaged emission spectra taken 30 seconds prior to sample application. This relative change could be entered into the mathematical model to determine analyte concentration.

To help meet the required accuracy and precision for an individual manufacturing lot, factory test data could be used in the mathematical model. The factory test data could be stored on either the test cartridge itself or on its packaging. Data storage could be done by means known in the art such as 1D barcodes, 2D barcodes, RFID tags and so forth.

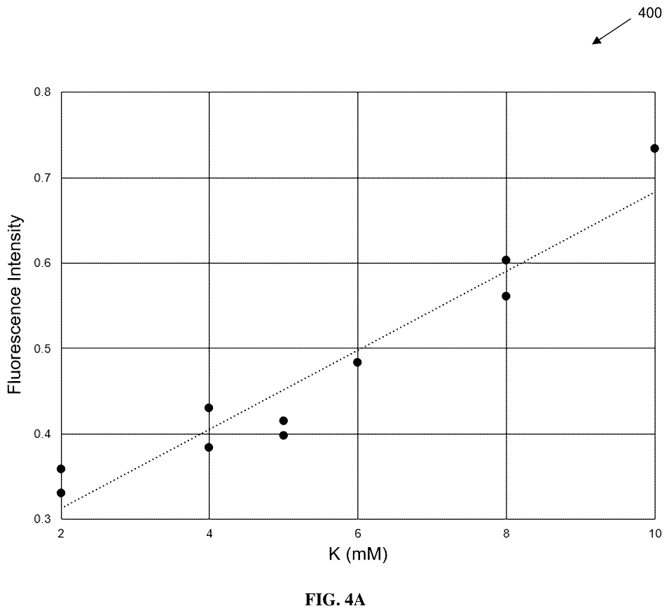

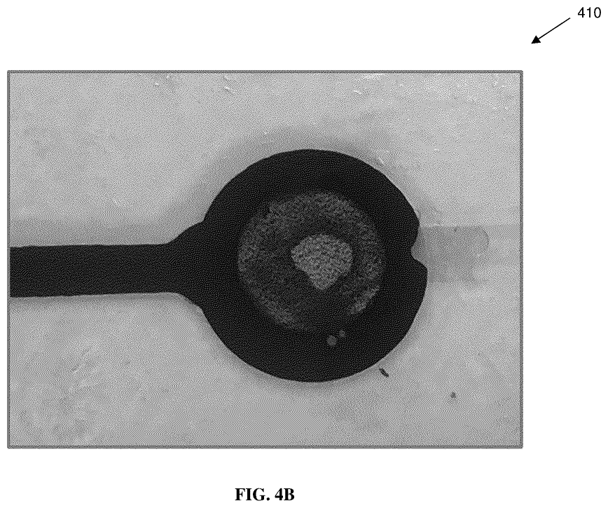

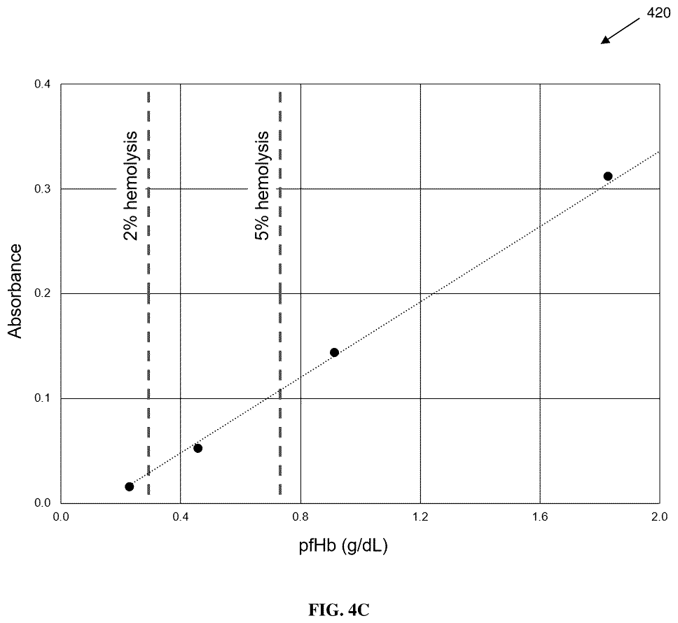

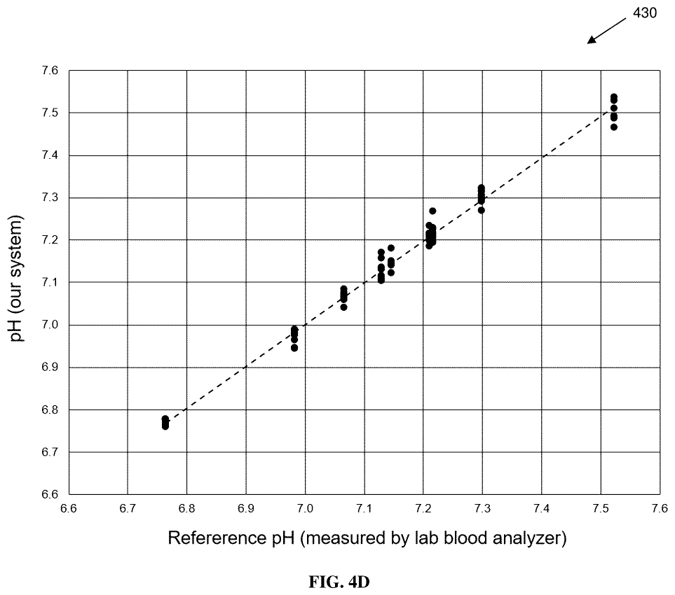

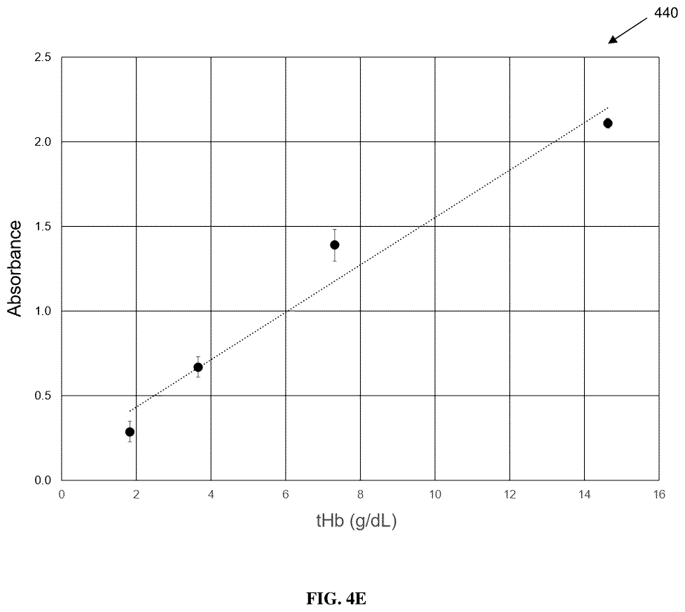

FIGS. 4A-4E illustrate experimental results obtained on our system. FIG. 4A is a diagram illustrating measurement data from the fluorescent K sensor. According to FIG. 4A, chart 400 shows the intensity of selected wavelengths of the fluorescence emission versus the K concentration in aqueous samples whereby the intensity of the emitted fluorescence varies linearly with the K concentration.

The measurement of the spectra across a sufficiently broad wavelength range, the sophistication of the mathematical model, the use of factory test data in that mathematical model, and the relatively small aging effects (i.e., low signal drift over time relative to electrochemical sensors) of fluorescent sensors all help contribute to a K sensor that does not need time-of-use calibration to deliver the required accuracy and precision. Eliminating the calibration fluid pack reduces the complexity and cost of the test cartridge.

The fluorescent K sensor is first created as a hydrogel suspension and then a small volume of this suspension is deposited or ‘printed’ directly onto the body of the test cartridge. FIG. 3B illustrates the print location for the single K sensor in the whole blood channel on the body of the test cartridge. The printed material is then dried to a certain extent; the drying process may be entirely passive or may be accelerated with heat and/or a reduction of the ambient relative humidity. The fluorescent K sensor is constructed from a single drop of the solution and does not require extra drops, or layers, printed over that first drop.

The manufacturing technique of printing the fluorescent K sensor directly onto the body of the test cartridge avoids the need to first print the fluorescent sensors onto a separate sensor substrate and then cutting, aligning and bonding the separate sensor substrate to the test cartridge. This reduces the complexity and cost of the test cartridge.

The test cartridge contains a plasma separation membrane to remove blood cells from the whole blood sample. FIG. 3D illustrates the location of the plasma separation membrane. The plasma separation membrane may be 330 μm thick polysulfone (such as Pall Vivid GR), but other materials and thicknesses may also be used. The plasma separation membrane is porous with hole sizes that are smaller than the size of blood cells; in this manner, the membrane may be used to filter out blood cells. Whole blood enters the membrane on one side, referred to as the blood-side surface, and plasma exits the membrane from the other side, referred to as the plasma-side surface.

The cartridge design illustrated in FIGS. 3A-3E uses a pre-manufactured module that contains the plasma separation membrane and adds this module to the body of the cartridge. The module contains the plasma separation membrane and 3 polymer layers. These elements may be bonded to one another by any means known in the art, including sensitive adhesives. The module may be pre-manufactured by a supplier and delivered in a format conducive to automated manufacturing, such as on a reel. Reeled modules closely resemble the reeled labels that are well known in the art and may permit the use of off-the-shelf label placing equipment to peel the modules from the reel and to place the modules into the body of the cartridge. As with labels, the face of the module that mates to the body of the cartridge may be covered in a pressure-sensitive adhesive.

The plasma separation region, illustrated in FIG. 3E, is designed to allow the movement of the whole blood onto substantially the entire blood-side surface of the plasma separation membrane before plasma separation substantially begins. If the whole blood is moved onto just a small portion of the blood-side surface of the plasma separation membrane, then that small portion may quickly clog with red blood cells, stopping the plasma separation process prematurely. Furthermore, any portion of the plasma separation membrane not filled with whole blood remains empty and tends to soak up separated plasma, preventing that separated plasma from leaving the plasma separation membrane and being used elsewhere in the cartridge. Therefore, movement of the whole blood onto substantially the entire blood-side surface of the plasma separation membrane before separation begins is required for successful plasma separation.

Movement of the whole blood onto substantially the entire blood-side surface of the plasma separation membrane is enabled by a circular channel surrounding the perimeter of the plasma separation membrane, as well as a cover layer that touches, but is not bonded to, the blood-side surface of the plasma separation membrane. This geometry is illustrated in FIG. 3E. The circular channel has a large cross-sectional area (approximately 1-2 mm wide and approximately the same 330 μm thickness as the plasma separation membrane) which allows fast flow of the whole blood, while the negligibly small gap between the blood-side surface of the plasma separation membrane and the cover layer has a negligibly small cross-sectional area and does not allow fast flow of the whole blood. In this manner, whole blood entering the plasma separation region first substantially fills the entire circular channel before flowing between the blood-side surface of the plasma separation membrane and the cover layer. The whole blood first substantially surrounding the entire perimeter of the plasma separation membrane ensures that the whole blood wets the blood-side surface of the plasma separation membrane from all directions and results in full wetting of substantially the entire blood-side surface of the plasma separation membrane.

This design confers a number of benefits. The first benefit is that very little, if any, of the plasma separation membrane remains unfilled with whole blood. This means that separated plasma is not wasted by soaking into any unfilled portions of the plasma separation membrane, which minimizes the volume of blood required to successfully run the test cartridge. Minimizing the volume of the blood sample that needs to be collected from the user is important as this increases the ease of use of the product and reduces the probability of extensive hemolysis during collection of a fingerstick blood sample. A second benefit of filling the plasma separation membrane using a circular channel around the perimeter of the plasma separation membrane, rather than over the blood-side surface of the plasma separation membrane, is that this design adds zero thickness to the cartridge, resulting in a thin cartridge that minimizes the complexity of the design. FIG. 3D illustrates the simplicity of using just 3 layers to create the entire plasma separation module: a spacer layer in the middle (the same thickness as the plasma separation membrane) that creates the circular channel around the perimeter of the plasma separation membrane and then a layer on each side. The simplicity of the design minimizes the number of cartridge components and minimises manufacturing complexity, which reduces the cartridge cost.

The flow speed of the whole blood into the circular channel must be kept above a certain minimum flow speed. Plasma separation membranes can exhibit significant capillary draw. A strong capillary draw may cause the whole blood to begin to flow between the blood-side surface of the plasma separation membrane and its cover layer before the whole blood has substantially surrounded the entire perimeter of the plasma separation membrane. In such cases, speeding up the flow of whole blood in the circular channel ensures that the whole blood substantially surrounds the entire perimeter of the plasma separation membrane before the whole blood flows significantly between the blood-side surface of the plasma separation membrane and the cover layer. Speeding up the flow rate of the whole blood in the circular channel may be accomplished by, for example, the use of negative pressure, or vacuum, applied downstream of the plasma separation module. To effect this, the instrument is provided with a pumping system that connects to the test cartridge upon insertion of the test cartridge into the instrument.

FIG. 4B is a diagram illustrating a successful plasma separation performed in-house using the perimeter blood-loading technique. According to FIG. 4B, photograph 410 shows a successful plasma separation using both the design geometry described above and the use of a vacuum to speed up the whole blood flow rate described above.

The pumping system connects to the air vent at the end of the single fluidic channel. The location of the air vent is illustrated in FIG. 3A and 3B. The pumping system applies a negative pressure. This negative pressure, or vacuum, may help pull the whole blood into the test cartridge, and/or move the whole blood over the whole blood fluorescent K sensor, and/or move the whole blood into the plasma separation membrane, and/or help draw the separated plasma away from the plasma separation membrane, and/or move the separated plasma into the plasma cuvette. Moving the whole blood into the plasma separation membrane is done at approximately 30 mbar vacuum, and drawing the separated plasma away from the plasma separation membrane is done at approximately 60 mbar vacuum, though other vacuum levels may be used. The test cartridge may contain a vent element, such as a hydrophobic porous plug, that allows air to pass but contains fluid inside the test cartridge.

The test cartridge contains a cuvette in the plasma channel, as illustrated in FIG. 3B. The cuvette is used to measure pfHb in plasma. The cuvette is filled with separated plasma and used to measure the transmission loss of light passing through that plasma. Cuvettes need to be relatively transparent to the transmitted light so that the cuvettes themselves do not excessively block transmission; for this reason, both the body of the test cartridge and the label used to close the cuvette will use materials that are relatively clear and transparent. Likely materials include polymers such as PETG, PMMA and COC.

The intensity of the transmitted light is first measured in the empty cuvette before the plasma arrives, then re-measured after the plasma has arrived and filled the cuvette. The change in light transmission, in conjunction with the known absorption spectrum of Hb, allows the calculation of the pfHb concentration. This concentration of free hemoglobin in the plasma can be used to estimate the degree of hemolysis in the whole blood sample and to judge whether the measured K levels in that same whole blood sample are substantially falsely elevated.

FIG. 4C is a diagram illustrating measurement data for pfHb concentration vs absorbance from a cuvette filled with plasma. According to FIG. 4C, chart 420 shows the light absorbance at selected wavelengths versus the pfHb concentration in plasma samples. Chart 420 illustrates experimental data plotted as light absorbance (calculated from the measured light transmission) versus the pfHb in plasma-filled cuvettes. The absorbance varies linearly with the pfHb concentration.

The test cartridge, or its packaging, may contain a means to store information. The information stored may include regulatory information such as test cartridge serial number, lot number, expiry date and unique device identifier (UDI) code. The information stored may also include factory test data such as measured calibration curves for the fluorescent sensors and measured optical path lengths for the cuvettes. The means of information storage are known in the art and include printed 1D barcodes, printed 2D barcodes, RFID tags and so forth.

The test cartridge contains a label, as illustrated in FIG. 3A. The label closes the fluidic channel and may also contain printed regulatory information such as test cartridge serial number, lot number, expiry date and UDI code. In addition, the label may contain printed 1D or 2D barcodes.

### Instrument

The instrument is configured to accept the test cartridge. The instrument comprises a source of excitation light for the fluorescent K sensor, a method to collect both the excitation light and the emitted fluorescent light and a means to measure the intensity of the excitation light and emitted fluorescent light across a certain wavelength range, providing either a single intensity measurement, or multiple measurements by wavelength, the latter resulting in an intensity spectrum and being accomplished by a device such as a spectrometer. The instrument also comprises a source of light for transmission or reflection through the cuvettes, a method to collect the transmitted light and a means to measure the intensity of the transmitted light across a certain wavelength range.

The instrument may include a means to read information stored on the test cartridge or its packaging. The means of information reading are known in the art and include an optical barcode reader and a RFID tag reader.

The instrument comprises a pumping system, including an air pump, optical sensors and pneumatic valves. The instrument comprises a means to connect the pumping system to the air vent of the test cartridge upon cartridge insertion into the instrument. The pumping system applies a negative pressure, or vacuum, to move the sample fluid though the test cartridge. Optical sensors may be used to detect the arrival of whole blood and plasma at key locations within the test cartridge; this information is used to trigger certain actions of the pumping operation. The pneumatic valves are used to switch the actions of the pumping operation from one mode to another; the modes may include venting the test cartridge to atmosphere, connecting the test cartridge to the air pump, and isolating the test cartridge from the air pump.

The instrument includes a computer processing means to convert the measured light intensities to analyte concentrations using proprietary mathematical models. These proprietary mathematical models may make use of any factory test data that may be stored on the test cartridge, or its packaging, such as measured factory calibration curves for the fluorescent sensors and measured optical path lengths for the cuvettes.

The instrument may comprise a screen to display information to the user; examples of the information that may be displayed include instrument status, instructions for use, measured analyte concentrations from the current test, and summaries of measured analyte concentrations from past tests. In some embodiments the screen may be a touch screen allowing the user to enter information via an on-screen keyboard; examples of the information that may be entered include patient details and physician details.

The instrument may comprise a means to transmit test records to software on a mobile device such as a mobile phone or a tablet computer. This mobile phone or tablet computer, in combination with said software, could take the place of the instrument in scanning data required for the test, such as patient information or factory test data mentioned above, using built-in optical and RFID scanners. If transmission between the instrument and phone/tablet is bidirectional, the phone/tablet can send information to the instrument that is required to perform a test. The means for transmission from the instrument to the device include methods known in the art such as Bluetooth and wi-fi. The screen of the mobile device may be used instead of and/or in addition to, the instrument screen to display and enter information.

FIG. 4D is a diagram illustrating measurement data from the fluorescent pH sensor vs lab blood analyzer in the same whole blood sample. According to FIG. 4D, chart 430 shows the pH measured by our system in whole blood samples versus the pH measured by a commercial laboratory blood analyzer in the same whole blood samples.

FIG. 4E is a diagram illustrating measurement data from cuvettes filled whole blood samples. According to FIG. 4E, chart 440 shows the light absorbance at selected wavelengths versus the total hemoglobin (tHb) concentration in whole blood samples.

According to the disclosure, a test cartridge configured for blood separation is disclosed. The test cartridge comprises a plasma separation element to separate plasma from whole blood. The test cartridge further comprises fluorescent sensors both upstream and downstream of the plasma separation element. The test cartridge further comprises one or more cuvettes both upstream and downstream of the plasma separation element.

According to the disclosure, the fluorescent sensors of the test cartridge are deposited directly onto the body of the test cartridge. The fluorescent sensors do not require time-of-use calibration. According to the disclosure, the measurements of the test cartridge are made using the transmission loss in cuvettes do not require time-of-use calibration.

According to the disclosure, the same blood sample of the test cartridge is used to measure potassium (K) in whole blood, then moved to the plasma separation element, then undergoes plasma separation, then measure hemolysis in the separated plasma so that all measurements come from the same blood sample and are directly correlated with one another.

According to the disclosure, the plasma separation membrane of the test cartridge is substantially loaded with whole blood by first substantially surrounding the periphery of the membrane with whole blood and only then allowing the whole blood to wet the blood-side surface of the membrane.

According to the disclosure, the plasma separation element of the test cartridge consists of 3 layers and a simple, off-the-shelf plasma separation membrane.

According to the disclosure, a method of separating whole blood from plasma, using a test cartridge and instrumentation is disclosed. The method comprises the steps of placing a whole blood sample on a sample port, filling a whole blood channel, measuring the whole blood with a first set of fluorescent sensors and a first cuvette, pulling the whole blood into a plasma separation membrane, pulling the separated plasma into the plasma channel, measuring the plasma with a second set of fluorescent sensors and a second cuvette and allowing the air in the whole blood channel and plasma channel to be displaced using an air vent.

According to the disclosure, blood of the method enters the test cartridge via an air pump or a hydrophilic membrane. According to the disclosure, the step of pulling the whole blood and separated plasma of the method is assisted with an air pump.

According to the disclosure, the method further comprises taking a measurement before and after the whole blood and plasma enter the test cartridge to measure relative change to a baseline. According to disclosure, the method further comprises an optical reader configured to measure the intensity of the light emitted from the fluorescent sensors at multiple wavelengths and configured to measure the intensity of the light transmitted or reflected through the cuvettes at multiple wavelengths.

According to the disclosure, a test cartridge configured for the measurement of potassium (K) and plasma free hemoglobin (pfHb) is disclosed. The test cartridge comprises a body frame, a sample port, an air vent port, a fluorescent potassium (K) sensor for making measurements in a whole blood channel, a plasma separation module, a cuvette for making plasma free hemoglobin (pfHb) measurements in a plasma channel.

According to the disclosure, the air vent is configured to allow air to be displaced out of the whole blood channel and the plasma channel and the use of an air pump in the instrument is configured to draw the whole blood from the sample port, across the fluorescent sensor, into the plasma separation module and draws the separated plasma into the cuvette.

According to the disclosure, the fluorescent sensor of the test cartridge can be printed in multiple layers wherein each layer is printed and dried before stacking the next layer on top. According to the disclosure, the plasma separation module of the test cartridge is constructed out of 3 layers.

The software on the mobile device may securely accept test record transmissions from the instrument. Furthermore, the software on the mobile device may facilitate the secure transmission of those test records to the patient's electronic medical record (EMR) so that test records may be shared with the patient's physician.

The software on the mobile device may include an interface allowing the physician to contact and communicate with the patient; such communication may be accomplished with a number of means known in the art, including texting, voice calling and video calling.

Although the disclosure has been described with reference to certain specific embodiments, various modifications thereof will be apparent to those skilled in the art without departing from the spirit and scope of the disclosure. All such modifications as would be apparent to one skilled in the art are intended to be included within the scope of the following claims.

Implementations disclosed herein provide systems, methods, and apparatus for generating or augmenting training data sets for machine learning training. The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code, or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database, or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

1. A test cartridge configured for blood separation comprising a plasma separation element to separate plasma from whole blood.

2. The test cartridge of claim 1, further comprising fluorescent sensors both upstream and downstream of the plasma separation element.

3. The test cartridge of claim 1, further comprising one or more cuvette both upstream and downstream of the plasma separation element.

4. The test cartridge of claim 2, wherein the fluorescent sensors have been deposited directly onto the body of the test cartridge.

5. The test cartridge of claim 2, wherein the fluorescent sensors do not require time-of-use calibration.

6. The test cartridge of claim 1, where the measurements made using the transmission loss in cuvettes do not require time-of-use calibration.

7. The test cartridge of claim 1, wherein the same blood sample is first used to measure potassium (K) in whole blood, then moved to the plasma separation element, then undergoes plasma separation, then measure hemolysis in the separated plasma so that all measurements come from the same blood sample and are directly correlated with one another.

8. The test cartridge of claim 1, where the plasma separation membrane is substantially loaded with whole blood by first substantially surrounding the periphery of the membrane with whole blood and only then allowing the whole blood to wet the blood-side surface of the membrane.

9. The test cartridge of claim 1, where the plasma separation element consists of 3 layers and a simple, off-the-shelf plasma separation membrane.

10. A method of separating whole blood from plasma, using a test cartridge and instrumentation, comprising the steps of:
placing a whole blood sample on a sample port;
filling a whole blood channel;
measuring the whole blood with a first set of fluorescent sensors and a first cuvette;
pulling the whole blood into a plasma separation membrane;
pulling the separated plasma into the plasma channel;
measuring the plasma with a second set of fluorescent sensors and a second cuvette; and
allowing the air in the whole blood channel and plasma channel to be displaced using an air vent.

11. The method of claim 10 wherein the blood enters the test cartridge via an air pump or a hydrophilic membrane.

12. The method of claim 10 wherein the step of pulling the whole blood and separated plasma is assisted with an air pump.

13. The method of claim 10 further comprising taking a measurement before and after the whole blood and plasma enter the test cartridge to measure relative change to a baseline.

14. The method of claim 10 further comprising an optical reader configured to measure the intensity of the light emitted from the fluorescent sensors at multiple wavelengths and configured to measure the intensity of the light transmitted or reflected through the cuvettes at multiple wavelengths.

15. A test cartridge configured for the measurement of potassium (K) and plasma free hemoglobin (pfHb), the test cartridge comprising:
a body frame;
a sample port;
an air vent port;
a fluorescent potassium (K) sensor for making measurements in a whole blood channel;
a plasma separation module;
a cuvette for making plasma free hemoglobin (pfHb) measurements in a plasma channel;
wherein the air vent is configured to allow air to be displaced out of the whole blood channel and the plasma channel; and
wherein the use of an air pump in the instrument is configured to draw the whole blood from the sample port, across the fluorescent sensor, into the plasma separation module and draws the separated plasma into the cuvette.

16. The test cartridge of claim 15 wherein the fluorescent sensor can be printed in multiple layers wherein each layer is printed and dried before stacking the next layer on top.

17. The test cartridge of claim 15 wherein the plasma separation module is constructed out of 3 layers.
